(19) United States
(12) Patent Application Publication
(10) Pub. No.: US 2011/0300102 A1
(43) Pub. Date: Dec. 8, 2011

(54) COMPOSITION FOR SKIN REGENERATION, CONTAINING A SECRETION IN THE CULTURE OF AN EMBRYONIC STEM CELL-DERIVED ENDOTHELIAL PROGENITOR CELL OR FRACTIONS THEREOF, AND USE THEREOF

(75) Inventors: Hyung Min Chung, Seoul (KR); Ji Mi Kim, Kyunggi-do (KR); Min Ji Lee, Seoul (KR); Ki Sung Hong, Seoul (KR); Jong Hyuk Sung, Kyunggi-do (KR)

(73) Assignee: CHA BIO & DIOSTECH CO., LTD., Kyunggi-do (KR)

(21) Appl. No.: 13/202,309

(22) PCT Filed: Feb. 23, 2010
(86) PCT No.: PCT/KR10/01101

§ 371 Date: Aug. 18, 2011

(30) Foreign Application Priority Data
Feb. 27, 2009 (KR) KR 10-2009-001722

(51) Int. Cl. A61K 38/20; A61K 8/64; A61P 17/02; A61Q 19/08; A61K 38/18; A61P 17/00

(52) U.S. Cl. 424/85.2; 514/8.1

(57) ABSTRACT

The present invention relates to a composition for skin regeneration, using a culture medium or a secretion in the culture of an embryonic stem cell-derived endothelial progenitor cell, and to the use thereof. As the present invention uses the secretion in the culture as a medicine and not the embryonic stem cell-derived endothelial progenitor cell itself, the risk of teratoma formation is prevented, and angiogenic activity is promoted to achieve improved effectiveness in healing wounds and burn wounds. The composition of the present invention, when used as a material in cosmetics, increases collagen synthesis and thus prevents skin aging. Particularly, the composition of the present invention in which the secretion in the culture is concentrated into a high concentration provides superior wound-healing effects as compared to a conventional human growth hormone (hGH).

### TECHNICAL FIELD

The present invention relates to a composition for skin regeneration, including a secretion in the culture of embryonic stem cell-derived endothelial progenitor cells (endothelial progenitor cells) or fractions thereof, and to the use thereof; more specifically, it relates to a technique for using biological proteins separated out from a culture medium of human embryonic stem-cell derived endothelial progenitor cells as a functional cosmetic product to improve wrinkles or prevent skin aging, which can be applied to wounds or wound burns, or can be applied to skin cosmetics as a treatment through the regeneration of skin.

### BACKGROUND ART

The majority of current treatments for healing wounds and healing burn wounds are broadly performed with a method of treatment through a therapeutic dressing gauze or through the transplantation of artificial skin. In the case of therapeutic dressing gauzes, the active treatment factors are absorbed in the gauze, wherein the factors are constituted of synthetic and natural macromolecules. There are useful aspects of the therapeutic dressing gauzes in that there is a reduced risk of infection with the transmission of the active factors, but there is also the possibility that the risk of infection may actually be increased in the event that a long-term dressing gauze is not regularly replaced, and there is also a concern that replacing the gauze will again irritate the site of the wound.

Artificial skin, which is applied to large burns or to surgical wounds, is constituted of synthetic macromolecules and natural macromolecules, such that usually the upper layer is provided with silicone in order to prevent loss of bodily fluids through evaporation and the lower layer is usually constituted of collagen or chondroitin sulfate in order to induce the regeneration of new blood vessels and connective tissue. The method in use is to take cells that constitute each layer of skin and expand the same in a test tube and then inoculate in an artificial skin scaffold made of appropriate synthetic macromolecules and natural molecules so as to induce the formation of tissues, which are then transplanted onto the patient. However, in terms of recovery from the wound, the current treatment methods that use artificial skin do not yield consistent results in wound healing; the results vary depending on whether the synthetic and natural macromolecules are biocompatible. In particular, there are difficulties with the cultured skin in terms of a stable supply of epidermal cells, as well as the risk of a rejection reaction from the immune system due to the immune-related components in the cells, such that joining the epidermis and dermis by grafting onto the wound site has unstable, critical shortcomings.

Normally, recovery from burns is achieved after the burn through processes including a coagulation phase, an inflammation phase, a cell migration phase, and a tissue remodeling phase, and is accompanied by effects such as angiogenesis, collagen synthesis and the migration of fibroblast cells from angiogenic effect of the various cytokines secreted in great quantities during the inflammation phase, such as EGF, FGF, VEGF, PDGF, TGF.

All these processes ultimately lead to an overall recovery from the wound because the different cytokines secreted from the localized wound site promote new angiogenesis, and because the various growth factors, inflammatory cells, stromal cells and dermal progenitor cells and the like that are required in wound recovery are loaded and carried by means of the regenerated blood vessels in order to promote the removal of tissue fragments and the formation of granulation tissue and the like.

The use of mesenchymal stem cell-derived secretions has already been reported to be effective in healing wounds. Stephen M et. al used bone marrow stem cell-derived endothelial progenitor cells to prove the efficacy thereof in treating wounds caused by ischemic diseases (Stephen M. Bauer, Lee J. Goldstein, Richard J. Bauer, Haiying Chen, Mary Putt, ScD, and Omaida C. Velazquez, The bone marrow-derived endothelial progenitor cell response is impaired in delayed wound healing from ischemia. Journal of vascular surgery (2006) 43(1):134-41), and Liwen Chen et al. used the secretions of bone marrow-derived mesenchymal stem cells, which are adult stem cells, to demonstrate the efficacy thereof in treating wounds (Liwen Chen, Edward E. Tredget, Philip Y. G. Wu, Yaojiong Wu, Paracrine Factors of Mesenchymal Stem Cells Recruit Macrophages and Endothelial Lineage Cells and Enhance Wound Healing(2008) PLoS ONE. April 2; 3(4):e1886). However, the yield of stem cells derived from the bone marrow, umbilical cord blood and the like of an adult is generally extremely low, the time during which subculturing is possible is also shorter than with other cell strains, and there is considerable difficulty in obtaining a large amount of secretions from the cell culture.

As an alternative thereto, it can be proposed that it may be possible to develop therapeutic agents for burns from human embryonic stem cell-derived endothelial progenitor cells, which have an infinite capacity for proliferation and differentiation. Human embryonic stem cells have been recognized to be very important cells in a variety of fields, being cells with a great deal of potential for treating incurable diseases, as has been announced in various studies that induced differentiation into vascular cells, nerve cells, muscle cells, pancreatic cells and the like (Thomson J A, Itskovitz-Eldor J, Shapiro S S, Waknitz M A, Swiergiel J J, Marshall V S, Jones J M, Embryonic stem cell lines derived from human blastocysts. Science (1998) 282:1145-1147) from stem cells, which possess an infinite capacity for self renewal (self-renewality) and, in special environments, pluripotency, which is the ability to differentiate into the triploblastic cells (of the endoderm, mesoderm and ectoderm) that make up the human body. However, histologically, a teratoma may form, which is a type of tumor that occurs during human embryonic stem cell differentiation from the intermixing of the tissues derived from the endoderm, mesoderm and ectoderm; very advanced technology is required in order to isolate the desired cells. Accordingly, there is an urgent need to develop therapeutic agents for wounds that are safer, which can be produced in large quantities, and for which the cost aspects are considered.

On the other hand, such culture secretions or a mixture of the constitutional factors thereof, which are effective in skin regeneration, could also be used as a novel cosmetic product for improving wrinkles or preventing skin aging.

### DETAILED DESCRIPTION OF THE INVENTION

### Technical Problems

The present invention was made to solve the problems described above and has the objective of providing a therapeutic composition and a method for treatment wherein there is no risk of teratomas while using embryonic stem-cell derived endothelial progenitor cells, and which is both safe and effective in regenerating skin. The present invention also has the additional objective of providing a composition that can be used as a cosmetic product for improving wrinkles or preventing skin aging by means of skin regeneration.

### Technical Method for Solving

In order to achieve the objectives above, the present inventors carried out various forms of research in order to develop a therapeutic agent for wounds and burn wounds using a concentrate from the secretions of human embryonic stem-cell derived endothelial progenitor cells and, as a result, discovered that when human embryonic stem-cell derived endothelial progenitor cells are cultured/replicated and the culture secretions are then obtained in large quantities, concentrated, and applied to animal models for wounds and burn wounds, the size of a wound site is reduced in a shorter period of time and more effectively in comparison to hGH (human growth hormone), which is currently widely used to heal wounds. Histological analyses of groups treated with the secretion concentrate from human embryonic stem-cell derived endothelial progenitor cells cultures indicate that the collagen layers contained more collagen than groups treated with the culture medium, and that re-epithelialization was more rapidly promoted in conjunction therewith, thus completing the present invention.

The present invention provides a composition for skin regeneration containing the secretions from a culture of embryonic stem-cell derived endothelial progenitor cells, or fractions thereof, as the active components.

The use of human embryonic stem cells as the above embryonic stem cells is included.

The culture secretions or fractions thereof are preferably concentrated to a high concentration, and are further preferably used at a concentration of 50 times or more.

The composition is effective in skin regeneration, and is preferably used in an application as a therapeutic agent for healing wounds, healing burn wounds or skin cosmetics treatment.

The composition can also be used for a cosmetic material with the function of improving wrinkles or preventing skin aging through skin regeneration.

At the same time, the present invention provides a composition for skin regeneration containing 1,250-1,250,000 pg/ml of epidermal growth factor (EGF), 35-35,000 pg/ml of basic fibroblast growth factor (FGF-2), 650-650,000 pg/ml of platelet-derived growth factor-AA (PDGF-AA), and 400-400,000 pg/ml of vascular endothelial growth factor (VEGF) as active components. The above composition can also be used for a cosmetic material having the function of improving wrinkles or preventing skin aging through skin regeneration.

The above composition may further contain one or more protein components selected from the group consisting of 4-4,000 pg/ml of Flt-3 ligand, 2-2,000 pg/ml of interleukin-1α (IL-1α), 2-2,000 pg/ml of interleukin-1β (IL-1β), 0.2-200 pg/ml of interleukin-17 (IL-17), 2-2,000 pg/ml of platelet-derived growth factor-BB (PDGF-BB), and 2-2,000 pg/ml of Rantes (CCL5).

The above composition may also further contain one or more protein components selected from the group consisting of 160-160,000 pg/ml fractalkine, 75-75,000 pg/ml granulocyte macrophage colony-stimulating factor (GM-CSF), 400-400,000 pg/ml of interleukin-6 (IL-6), 19,000-19,000,000 pg/ml of interleukin-8 (IL-8), 34-34,000 pg/ml of interleukin-9 (IL-9), 45-45,000 pg/ml of chemokine IP-10, and 6-6,000 pg/ml of monocyte chemoattractant protein-1 (MCP-1).

The present invention further provides a method for treatment using the above composition for skin regeneration.

Such a method for treatment is performed so as to include a step (a) for culturing the embryonic stem-cell derived endothelial progenitor cells; a step (b) for removing the endothelial progenitor cells so as to prepare the culture secretions or fractions thereof, and a step (c) for applying the culture secretions or fractions thereof to the skin of a mammal.

The method for treatment preferably further includes a step for concentrating the culture secretions or fractions thereof 50 times or more.

In order to effectively acquire the secretions from the human embryonic stem-cell derived endothelial progenitor cells culture in step (a), the culturing is preferably done after the culture dish has been coated with collagen, and culturing is also preferably done using an EGM-2 MV culture medium as the culture medium, although it is possible to use other known culture mediums according to need, such as a DMEM culture medium, an M199 culture medium, or a culture medium that is a mixture thereof; the cell proliferation can be bred by subcultures, and the respective culture mediums required for the different subculturing steps may be selected in order to perform the culturing.

Also, in order to concentrate the resulting culture secretions to a high degree of concentration, a concentration system using a TFF membrane may be used, wherein it is preferable to make the concentrate by concentrating only those substances that are 10 KDa or more, but it is also possible to use a concentrate of those substances that are 3 KDa or 5 KDa, or of all the substances, according to need.

In order to utilize the concentrate of the secretions from the culture of embryonic stem cell-derived endothelial progenitor cells in Step (b) above, a silicone ring may be fitted to the wound site, following which the concentrate of the secretions from the culture of embryonic stem cell-derived endothelial progenitor cells is applied to the interior thereof.

Human embryonic stem cells may be used as the embryonic stem cells; the human embryonic stem cells have an infinite capacity to proliferate and can therefore be continuously cultured by means of subcultures, and may be provided as a composition that can be distributed using a known method such as freeze-drying. The concentrate may be provided as a commercial product that can be distributed as a concentrate having a high degree of concentration wherein a TFF membrane has been used so as to concentrate the same and to which preservatives, stabilizers and the like have been added.

### Advantageous Effects

Because human embryonic stem cells, which have an infinite autoproliferative ability, are utilized as the supply source in the skin-regenerative composition of the present invention and the method for treating wounds and burn wounds using the same, not only can the problem of a restricted supply of cells be greatly reduced, but also risks such as the formation of teratomas are absent due to the fact that only the culture secretions are used as the therapeutic agent, instead of transplanting the cells themselves. On the other hand, a cosmetic composition which contains the skin-regenerative composition of the present invention has the effect of providing supple skin by both improving wrinkles and preventing skin aging.

In particular, with regards to the course after application of the concentrate of secretions from a culture of human embryonic stem-cell derived endothelial progenitor cells according to the present invention, it has been found that the size of the wound site is reduced in a shorter period of time compared to comparison groups with human growth hormone (hGH) and the like, and also that re-epithelialization is effectively promoted by the formation of multiple collagen layers. Moreover, it is possible to more effectively treat wounds, due to the angiogenesis-promoting and wound-healing factors that are contained in the concentrate of secretions from a culture of human embryonic stem-cell derived endothelial progenitor cells applied according to the treatment method of the present invention, because the wound-healing factors found in animal bodies (growth factors, inflammation cells, stromal cells, dermal progenitor cells and the like) migrate more easily to the wound site.

Accordingly, there is no restriction on the supply of cells in the method for treating wounds of the present invention, and the culture secretions can be obtained in large quantities, such that a wound can be treated in a shorter period of time and with a higher efficacy than in conventional methods for treating wounds that use human growth hormone (hGH).

### BRIEF DESCRIPTION OF THE DRAWINGS

FIG. 1 is a table wherein a multiplex cytokine array of the concentrate of secretions from the culture of human embryonic stem-cell derived endothelial progenitor cells of the present invention was used to analyze the components and the active ingredient contents of the concentrate of the secretions of the culture of human embryonic stem-cell derived endothelial progenitor cells.

FIG. 2 is a diagram with photographs showing the process for preparing and applying the animal wound models in order to verify efficacy in treating wounds.

FIG. 3a shows the measurements of the size of healing from a wound carried out on day 14 with wound models treated with low-concentration secretions from a human embryonic stem-cell derived endothelial progenitor cell culture (1×), a concentrate of highly-concentrated secretions from a human embryonic stem-cell derived endothelial progenitor cell culture (50×), a concentrate of highly-concentrated secretions from a human embryonic stem-cell derived endothelial progenitor cell culture (50×) with hGH, and hGH alone.

FIG. 3b shows the results from a histological analysis of the animal models; when tissue was taken from the wound site and stained with Masson's trichrome stain, the results confirmed collagen synthesis.

FIG. 4 is a diagram with photos showing the steps for preparing animal burn wound models in order to verify efficacy in improving the treatment of burn wounds; the reduction in the size of the wound in each group was observed for two weeks in order to verify improvement efficacy.

FIG. 5a shows the measurements of the size of healing from a wound with burn wound models treated for 14 days with low-concentration secretions from a human embryonic stem-cell derived endothelial progenitor cell culture, a concentrate of highly-concentrated secretions from a human embryonic stem-cell derived endothelial progenitor cell culture (50×), a concentrate of highly-concentrated secretions from a human embryonic stem-cell derived endothelial progenitor cell culture (50×) with hGH, and hGH alone.

FIG. 5b shows the results from a histological analysis of the animal models; when tissue was taken from the wound site and stained with Masson's trichrome stain, the results confirmed collagen synthesis.

FIG. 6a shows photographic results from testing of cellular activity of human dermal fibroblast cells (HDF) by UV irradiation in which the control group was not treated with the composition of the present invention; in the photographic results of Embodiment 2 and Embodiment 3, the compositions according to the respective embodiments of the present invention were administered.

FIG. 6b shows the cell number of human dermal fibroblast cells (HDF) after UV irradiation according to the control group and Embodiments 2 and 3 of FIG. 6a.

FIG. 7 shows the results of a western blot analysis for treatment with the compositions of Embodiments 2 and 3 of the present invention.

### MODES FOR CARRYING OUT THE INVENTION

In this specification, “embryonic stem cells” includes all mammal-derived embryonic stem cells, including human embryonic stem cells.

“Human embryonic stem cells” includes totipotent cells derived from the inner cell pass of a human morula. For example, CHA-hES3 (Jumi Kim, Sung-Hwan Moon, Soo-Hong Lee, Dong-Ryul Lee, Gou-Young Koh, and Hyung-Min Chung, Effective Isolation and Culture of Endothelial Cells in Embryoid Body Differentiated from Human Embryonic Stem cells (2007) STEM CELLS AND DEVELOPMENT 16:269.280) and the like can be used, but the human embryonic stem cells are not to be limited to this example. In addition, human embryonic stem cells can be constructed easily by one skilled in the art.

It is preferable to establish and culture embryonic stem cell-derived endothelial progenitor cells concentrated to a high degree of concentration by parenchymal cell sorting (FACS) using a CD133/KDR double marker, after the human embryonic stem-cell derived endothelial progenitor cells used to obtain the culture secretions are induced into differentiation by the formation of an embryoid body under hypoxic conditions.

In the present invention, “a concentrate of secretions of a culture of human embryonic stem-cell derived endothelial progenitor cells” signifies culture secretions obtained from endothelial progenitor cells derived from human embryonic stem cells, and includes highly-concentrated concentrates wherein the same has been concentrated.

Below is provided a more detailed description of the present invention by means of embodiments. However, these embodiments are examples of the present invention for illustrative purposes only, and the scope of the present invention is not to be limited to these embodiments.

### Embodiment 1

(1) Acquisition and Concentration of Human Embryonic Stem-Cell Derived Endothelial Progenitor Cell Culture Secretions

Human embryonic stem-cell derived endothelial progenitor cells were cultured in the culture medium EGM-2/MV (Cambrex) on a culture dish coated with collagen; once the cells were distributed in the culture dish at a concentration of about 70% to 80%, the concentrate of secretions from the human embryonic stem-cell derived endothelial progenitor cells culture was obtained through a 48-hour culturing. The resulting culture secretions were concentrated 50 times using a TFF membrane concentration system in order to obtain the highly-concentrated culture secretion concentrate.

(2) Preparing Animal Wound Models and Animal Burn Wound Models

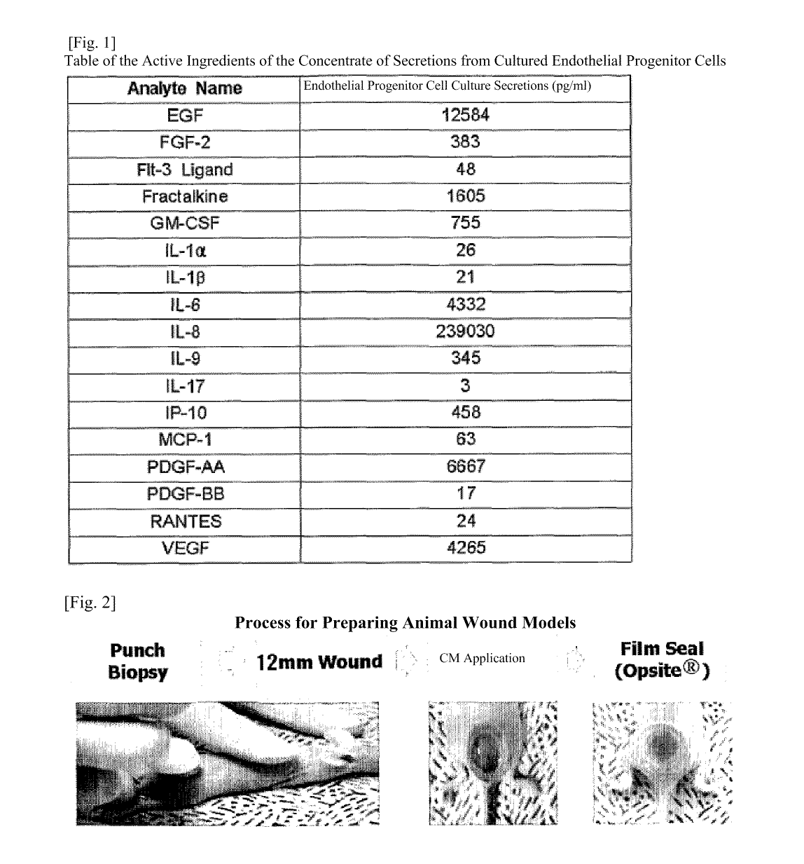

FIG. 2 is a diagram with photographs showing the process for preparing and using the animal wound models for verifying efficacy in treating wounds.

To prepare the animal wound models for verifying efficacy in treating wounds, the animal models were prepared after a dorsal biopsy punch was used to induce a 12 mm transmural ablation wound in the skin of 6-week-old nude mice, and a silicone ring was placed at the wound site in order to continuously expose the wound site to the culture secretion concentrate during use.

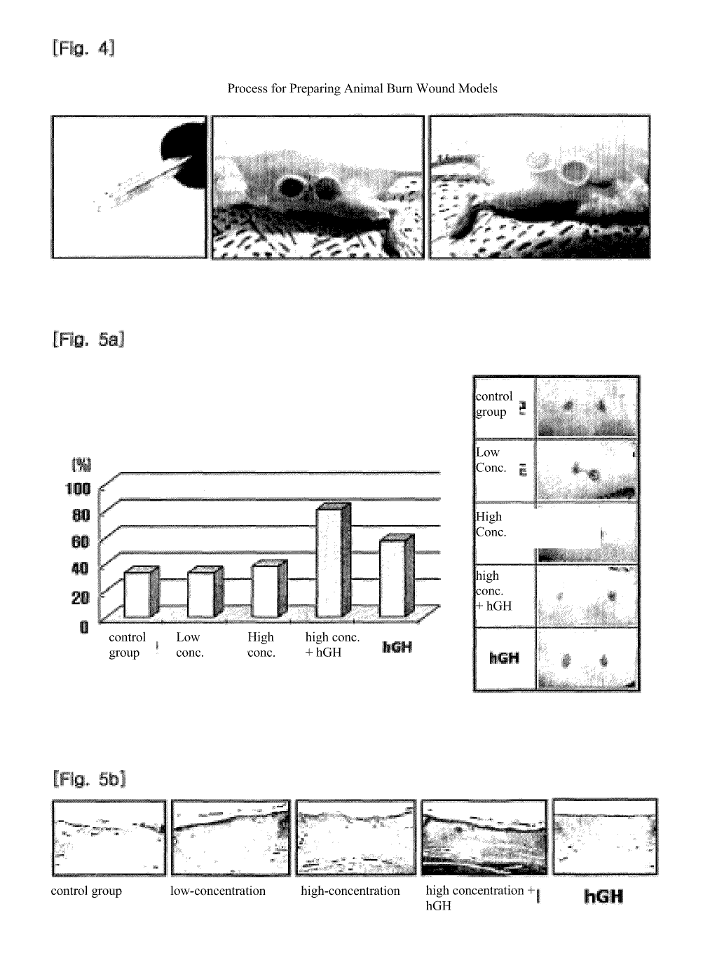

FIG. 4 is a diagram with photos showing the steps for preparing animal burn wound models in order to verify efficacy in improving the treatment of burn wounds; the reduction in the size of the wound in each group was observed for two weeks in order to verify improvement efficacy.

To prepare the animal burn wound models for verifying efficacy in treating burn wounds, a method was used wherein an iron stamp was lightly placed on the backs of 6-week-old CF-1 mice, a 5 mm transmural burn wound was induced in the skin for 10 seconds at 70°. After the burn was dressed, the remaining heat was removed from the tissue water using an ice pack and the burn wound site was then cooled for about five minutes, thus preparing the animal models.

(3) Applying the Human Embryonic Stem-Cell Derived Endothelial Progenitor Cell Culture Secretion Concentrate to the Animal Wound Models and Wnimal Burn Wound Models

The obtained concentrate of the secretions from the human embryonic stem-cell derived endothelial progenitor cell culture was then applied to the prepared animal wound models. During the application, 200 μL of the concentrate of the secretions from the human embryonic stem-cell derived endothelial progenitor cell culture were applied within the silicone ring on the animal wound models.

In order to verify the efficacy of the concentrate of secretions from the human embryonic stem-cell derived endothelial progenitor cell culture, the culture medium (EGM-2 MV medium), low-concentration secretions from the culture of human embryonic stem cell-derived endothelial progenitor cells, or human growth hormone (hGH) were applied to the same animal wound models as comparison groups, and then the degree to which the size of the wound was reduced was observed for two weeks.

In addition, a histological analysis was conducted after week 2 to observe collagen synthesis and the progress of re-epithelialization, which are important factors in healing wounds. Masson's trichrome staining was carried out to confirm collagen synthesis.

### Experimental Example 1

A multiplex cytokine array was used on the concentrate of secretions from a human embryonic stem-cell derived endothelial progenitor cell culture obtained after culturing under the conditions set forth in (1) of Embodiment 1 in order to analyze the active ingredients in the resulting highly-concentrated concentrate (50 times).

FIG. 1 is a table showing an analysis of the components and amount of active ingredients in the concentrate of secretions from the human embryonic stem-cell derived endothelial progenitor cell culture of the present invention.

As shown in FIG. 1, the concentrate of secretions from the human embryonic stem-cell derived endothelial progenitor cell culture, concentrated 50 times, was found to contain large amounts of active ingredients that promote angiogenesis and wound healing, such as EGF, FGF-2, Fractalkine, GM-CSF, IL-6, IL-8, IL-9, IP-10, MCP-1, PDGF-AA, PDGF-BB, and VEGF.

The above results show that the concentrate of secretions from a human embryonic stem-cell derived endothelial progenitor cell culture used in the present invention contains large quantities of the active ingredients promoting angiogenesis and wound healing, thus facilitating healing from wounds and burn wounds.

### Experimental Example 2

After the method set forth in (2) of Embodiment 1 was used to prepare animal wound models, the concentrate of secretions from a human embryonic stem-cell derived endothelial progenitor cell culture and the other comparison groups were applied inside the dorsal silicone ring (see FIG. 2).

### Experimental Example 3

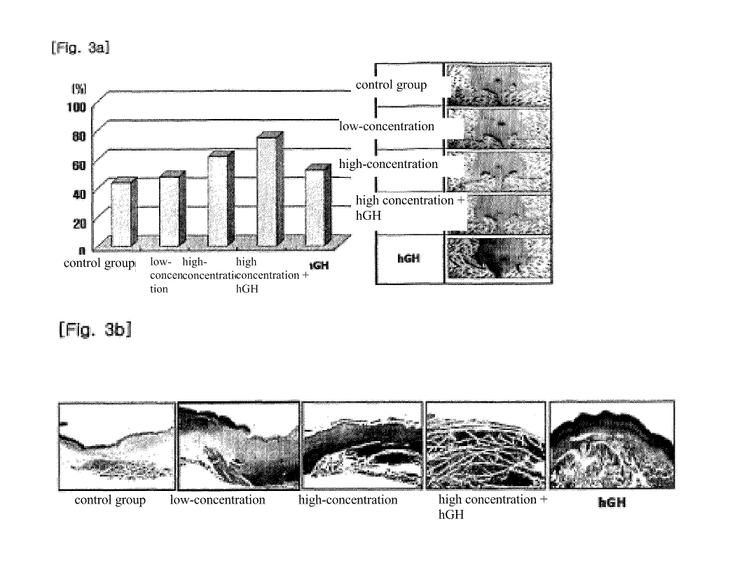

As set forth in (3) of Embodiment 1, the culture medium (EGM-2 MV medium; the control group), low-concentration secretions from the culture of human embryonic stem cell-derived endothelial progenitor cells (low concentration), high-concentration secretions from the culture of human embryonic stem cell-derived endothelial progenitor cells (high concentration; 50 times), and human growth hormone (hGH) were each applied, and then each treated group was observed for two weeks in order to observe the degree to which the size of the wound was reduced (see FIG. 3a). As shown in FIG. 3a, the wound size reduction in the culture medium treated group (Medium) took place later on, while a somewhat more rapid wound size reduction was observed in the group treated with low-concentration human embryonic stem cell-derived endothelial progenitor cell culture secretions (low-concentration) and in human growth hormone than in the control group treated with the culture medium, but in the case of the group treated with a human embryonic stem-cell derived endothelial progenitor cell culture secretion concentrate (high-concentration) and the composite group treated with the human embryonic stem-cell derived endothelial progenitor cell culture secretion concentrate and human growth hormone (hGH), a markedly more rapid reduction in wound size was observed than the other treated groups.

The above results show that the efficacy in healing wounds when the concentrate of secretions from a human embryonic stem-cell derived endothelial progenitor cell culture as used in the present invention is far more efficient than the other treated groups.

### Experimental Example 4

As set forth in (3) of Embodiment 1, a concentrate of secretions from human embryonic stem-cell derived endothelial progenitor cells was applied to prepare animal wound models and a histological analysis was carried out at week 2 by removing samples from the wound sites.

As is verified in FIG. 3b, markedly more collagen synthesis was confirmed in the group to which the concentrate of secretions from a human embryonic stem-cell derived endothelial progenitor cell culture (high concentration) was applied to be treated according to the treatment method of the present invention than in the comparison groups treated with the culture medium, and similarly, markedly more collagen synthesis was also confirmed in the composite group treated with the concentrate of secretions from a human embryonic stem-cell derived endothelial progenitor cell culture and human growth hormone (hGH) (high concentration+hGH).

The above results show that treating wounds using the concentrate of secretions from a culture of human embryonic stem-cell derived endothelial progenitor cells as used in the present invention results in synthesis of a high quantity of collagen, which is essential in wound healing, and the collagen synthesis plays a key role in re-epithelialization and promotes rapid re-epithelialization. The synthesis of a high quantity of collagen indicates that the skin-regenerative composition of the present invention can be used as a cosmetic product for improving wrinkles or preventing skin aging.

### Experimental Example 5

After animal burn wound models were prepared using the method as set forth in (2) of Embodiment 1, the concentrate of secretions from the culture of human embryonic stem-cell derived endothelial progenitor cells and comparison groups were applied to the dorsal burn wound sites of the animal burn wound models (see FIG. 4).

### Experimental Example 6

As set forth in (3) of Embodiment 1, the culture medium (EGM-2 MV medium; the control group), low-concentration secretions from the culture of human embryonic stem cell-derived endothelial progenitor cells (low concentration), high-concentration secretions from the culture of human embryonic stem cell-derived endothelial progenitor cells (high concentration; 50 times), and human growth hormone (hGH) were each applied, and then each treated group was observed for two weeks in order to observe the degree to which the size of the wound was reduced (see FIG. 5a). As shown in FIG. 5a, wound size reduction was slowest in the control group, while a somewhat more rapid reduction in wound size was observed in the cases of the low-concentration, the high-concentration and human growth hormone (hGH), which were the comparison groups, than in the control group. A markedly more rapid reduction in wound size was observed in the case of high-concentration+hGH than in the control group and the other comparison groups.

The above results show that the efficacy of using the human embryonic stem-cell derived endothelial progenitor cell secretions to enhance burn wound treatment as used in the present invention is far more efficient than in the control group.

### Experimental Example 7

As set forth in (3) of Embodiment 1, human embryonic stem-cell derived endothelial progenitor cells were applied to animal wound models and then a histological analysis was conducted by removing samples from the wound sites after week 2.

As can be confirmed from FIG. 5b, synthesis of significantly more collagen was confirmed in the groups treated with human embryonic stem-cell derived endothelial progenitor cell secretions applied according to the treatment method of the present invention than in the control group.

The above results show that using human embryonic stem-cell derived endothelial progenitor cells as used in the present invention results in improved burn wound healing due to the synthesis of a high quantity of collagen, which is essential in wound healing, and the synthesis of collagen plays a key role in re-epithelialization and promotion of rapid re-epithelialization. The synthesis of a high quantity of collagen indicates that the skin-regenerative composition of the present invention can be used as a cosmetic product for improving wrinkles or preventing skin aging.

### Embodiment 2

A composition for improving wrinkles or preventing skin aging was prepared by mixing epidermal growth factor (EGF), (basic) fibroblast growth factor-2 (FGF-2), platelet-derived growth factor-AA (PDGF-AA), and vascular endothelial growth factor (VEGF) at the concentrations shown in Table 1 below into the components of Embodiment 1.

TABLE 1

| Component | Concentration |
|---|---|
| name | (pg/ml) |
| EGF | 11245 |
| FGF-2 | 395 |
| PDGF-AA | 5745 |
| VEGF | 4185 |

### Embodiment 3

A composition for improving wrinkles or preventing skin aging was prepared by mixing epidermal Growth Factor (EGF), (basic) fibroblast growth factor-2 (FGF-2), platelet-derived growth factor-AA (PDGF-AA), vascular endothelial growth factor (VEGF), Flt-3 ligand, interleukin-1α (IL-1α), interleukin-1β (IL-1β), interleukin-17 (IL-17), platelet-derived growth factor-BB (PDGF-BB), and (Rantes, CCL5) at the concentrations shown in Table 2 below into the components of Embodiment 1.

TABLE 2

| Component | Concentration |
|---|---|
| name | (pg/ml) |
| EGF | 11245 |
| FGF-2 | 395 |
| PDGF-AA | 5745 |
| VEGF | 4185 |
| Flt-3 Ligand | 40 |
| IL-1α | 23 |
| IL-1β | 22 |
| IL-17 | 3 |
| PDGF-BB | 20 |
| RANTES | 21 |

### Experimental Example 8

### Testing the Cellular Activity of Human Dermal Fibroblast Cells (Evaluation of Wrinkle Activity)

Human dermal fibroblast cells (HDF) were added to a batch of DMEM (Invitrogen-Gibco-BRL, Grand Island, N.Y.) supplemented with 10% bovine serum, 100 U/mL of penicillin and 100 μg/mL streptomycin and then cultured under conditions of 37° C. and 5% CO2. [101] The cultured HDF cells were then transplanted into well plates using cell batches of 5D10⁴ per well in order to treat the cultures prepared in Embodiments 2 and 3 for up to 24 hours, and then washed with a phosphate buffer solution. Thereafter, UV irradiation was used and after the UV irradiation (UVA: 10 J/cm²), the batches were inserted and cultured for 72 hours. A CCK-8 kit (Dojindo, Gaithersburg, Md., USA) was used to initiate a reaction at 37° C. for two hours, and microplate reader devices were used to measure the absorbance thereof at 450 nm; photographs were taken to compare whether there was cell growth.

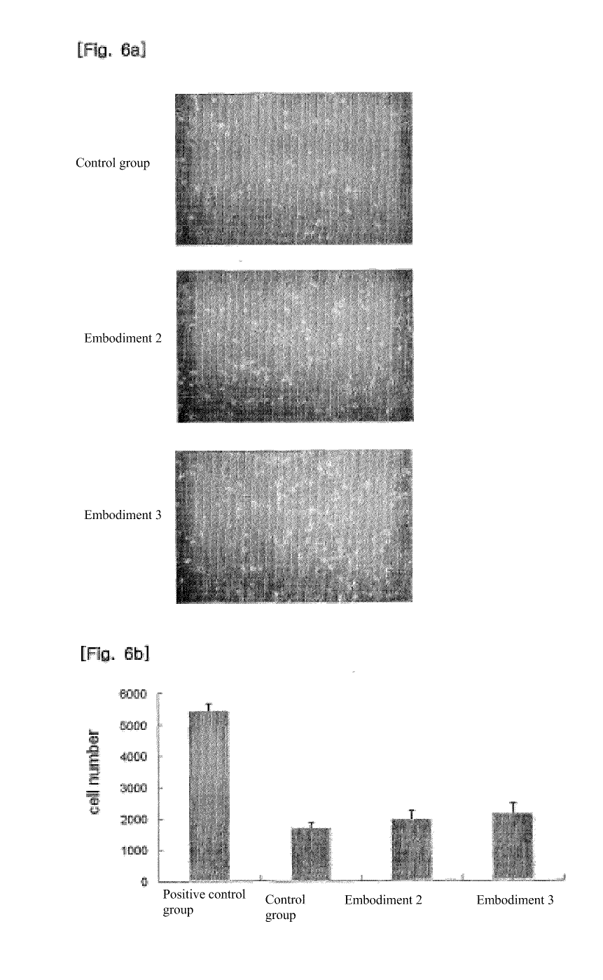

In FIG. 6a, the control group is a photograph of when the composition of the present invention was not treated, while Embodiments 2 and 3 are photographs of when the compositions according to the embodiments of the present invention were treated.

FIG. 6b shows the cell count of human dermal fibroblast cells (HDF) after UV irradiation.

As shown in FIGS. 6a and 6b, it is confirmed that the protein composition prepared in Embodiment 2 had significantly more activity in the HDF cells damaged by UV when compared with the control group, and it was confirmed that Embodiment 3 also had marked activity.

### Experimental Example 9

### Confirmation of Collage Expression (Western Blot Analysis)

After the cultured HDF cells were transplanted in batches of 2D10⁵ to the well plates, the compositions prepared in Embodiments 2 and 3 were treated for up to 24 hours and then washed with a phosphate buffer solution. Thereafter, UV irradiation was used and after the UV irradiation (UVA: 10 J/cm²), the batches of DMEM were inserted and cultured for 72 hours. After being washed for 72 hours with the phosphate buffer solution, the cells were lysed using a RIPA buffer solution (50 mm tris-HCl, 0.15 M NaCl, 1 mM EDTA, 1% Triton X-100, 1% SDS, 50 mM NaF, 1 mM Na₃PO₄, 5 mM dithiothreitol, 1 μg/mL leupeptin and 20 μg/mL PMSF, pH 7.4) and the proteins were collected. 8% SDS-polyacrylamide gel electrophoresis was used to separate out 20 mg of protein. The gel from which the protein was separated out was transferred to a PVDF film and the PVDF film was reacted with anti-collagen antibodies (dilution ratio, 1:250) and then re-reacted using horseradish peroxidase-conjugated anti-goat IgG antibody (dilution ratio, 1:10,000). The resulting bands were analyzed by exposure on X-ray film using immunobilon western reagent.

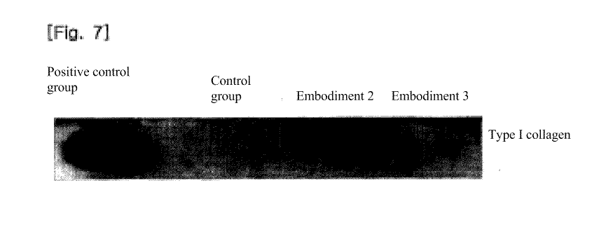

FIG. 7 shows the results of a western blot analysis for when the compositions of Embodiments 2 and 3 of the present invention are treated. As can be seen in FIG. 7, no collagen expression was exhibited after UV irradiation in the control group wherein the composition of the present invention was not treated, whereas collagen was expressed when the composition of Embodiment 2 was treated, and a significant increase in collagen expression was confirmed when the composition of Embodiment 3 was treated.

### INDUSTRIAL APPLICABILITY

The present invention relates to a composition for skin regeneration, containing a secretion in the culture of embryonic stem cell-derived endothelial progenitor cells or fractions thereof, and to the use thereof; it also relates to a technique for using biological proteins separated out from a culture medium of human embryonic stem-cell derived endothelial progenitor cells as a cosmetic product with the functions of improving wrinkles or preventing skin aging, which can either be applied to wounds or wound burns, or can be applied to skin cosmetics as a treatment through the regeneration of skin.

1. A composition for skin regeneration, comprising secretions from a culture of embryonic stem-cell derived endothelial progenitor cells or fractions thereof.

2. The composition for skin regeneration according to claim 1, wherein the embryonic stem cells are human embryonic stem cells.

3. The composition for skin regeneration according to claim 1, characterized in that the culture secretions or fractions thereof are concentrated 50 times or more.

4. The composition for skin regeneration according to claim 1, wherein the composition is used in healing wounds, healing burn wounds, or skin cosmetics.

5. The composition according to claim 1, wherein the composition is used in a cosmetic product for improving wrinkles or preventing skin aging.

6. A composition for skin regeneration, comprising 1,250-1,250,000 pg/ml of epidermal growth factor (EGF), 35-35,000 pg/ml of (basic) fibroblast growth factor (FGF-2), 650-650,000 pg/ml of platelet-derived growth factor-AA (PDGF-AA), and 400-400,000 pg/ml of vascular endothelial growth factor (VEGF).

7. The composition for skin regeneration according to claim 6, further comprising one or more proteins selected from the group consisting of 4-4,000 pg/ml of Flt-3 ligand, 2-2,000pg/ml of interleukin-1α (IL-1α), 2-2,000 pg/ml of interleukin-1β (IL-1β), 0.2-200 pg/ml of interleukin-17 (IL-17), 2-2,000 pg/ml of platelet-derived growth factor-BB (PDGF-BB), and 2-2,000 pg/ml of Rantes (CCL5).

8. The composition for skin regeneration according to claim 6, further comprising one or more proteins selected from the group consisting of 160-160,000 pg/ml of fractalkine), 75-75,000 pg/ml of granulocyte macrophage colony-stimulating factor (GM-CSF), 400-400,000 pg/ml of interleukin-6 (IL-6), 19,000-19,000,000 pg/ml of interleukin-8 (IL-8), 34-34,000 pg/mi of interleukin-9 (IL-9), 45-45,000 pg/ml of chemokine IP-10, and 6-6,000 pg/ml of monocyte chemoattractant protein-1 (MCP-1)

9. The composition for skin regeneration according to claim 6, wherein the composition is used in healing wounds, healing burn wounds or in skin cosmetics.

10. The composition according to claim 6, wherein the composition is used in a cosmetic product for improving wrinkles or preventing skin aging.

11. A method for healing wounds, healing burn wounds, or skin cosmetics, comprising
(a) a step for culturing embryonic stem-cell derived endothelial progenitor cells;
(b) a step for removing the endothelial progenitor cells and preparing the secretions from the culture or fractions thereof; and
(c) a step for applying the culture secretions or fractions thereof to the skin of a mammal.

12. The method for healing wounds, healing burn wounds, or skin cosmetics according to claim 11, further comprising a step for concentrating the culture secretions or fractions thereof 50 times or more.

13. The method for healing wounds, healing burn wounds, or skin cosmetics according to claim 12, characterized in that the step for concentration uses a concentration system using a TFF membrane in order to concentrate only those components that are 10 KDa or more.
